(19) United States
(12) Patent Application Publication
(10) Pub. No.: US 2012/0165236 A1
(43) Pub. Date: Jun. 28, 2012

(54) CLEANSING AGENT

(75) Inventor: Shoichi Nakamura, Higashichikuma-gun (JP)

(73) Assignees: Shoichi NAKAMURA, Hagashichikuma-gun (JP); ACP JAPAN CO., LTD., Bunkyo-ku (JP)

(21) Appl. No.: 13/138,936

(22) PCT Filed: Jul. 23, 2010
(86) PCT No.: PCT/JP2010/062441

§ 371 Date: Oct. 27, 2011

(51) Int. Cl. A61K 8/73; A61Q 1/14; A61Q 19/10; C11D 7/60; A61Q 5/02

(52) U.S. Cl. 510/119; 510/470; 510/463; 510/130; 510/138; 510/136; 510/137

(57) ABSTRACT

To prevent a photocatalyst-containing cleansing agent from deteriorating and maintain the sterilizing cleansing and deodorant effects, provided is a cleansing agent characterized by containing a photocatalyst, fine powder of charcoal that is activated carbon, and cyclodextrin. It is desirable that the charcoal is charcoal or activated carbon. By adding a fine powder of charcoal in addition to the photocatalyst, it is possible to inhibit the oxidation reaction of the photocatalyst during storage by the light blocking effect, and to prevent the effects of the cleansing agent from degrading. Further, it is possible to efficiently exert cooperative action of adsorbing excess sebum, pore-clogging dirt, cuticle, waste products, smells, etc. due to the porous structure of charcoal, and further, decomposing the adsorbed materials by the photocatalyst.

### TECHNICAL FIELD

The present invention relates to cleansing agents for preventing photocatalyst-containing cleansing agents from deteriorating and enabling the sterilizing, cleansing and deodorant effects to be maintained.

### BACKGROUND ART

Conventionally, among cleansing agents, particularly, components contained in cleansing agents (for example, shampoos) used for the human body are required to be gentler to the skin and mucous membrane because the agents directly contact the human body, and further, to have great effects in bactericidal activity, cleansing activity, deodorant activity, etc.

Then, cleansing agents with photocatalysts added thereto have previously been proposed (for example, see Patent Document 1).

The photocatalyst is a generic name for substances exhibiting the catalyst action by being irradiated with light, and titanium oxide is known as a representative example. The photocatalyst has the action of decomposing organic substances by oxidation-reduction action when being irradiated with light. Accordingly, when the photocatalyst is added to the cleansing agent, it is possible to remove bacteria, sebum, bad smells, etc. As a result, for example, when the cleansing agent is a shampoo, it is possible to obtain the action and effect of hair growth, etc.

### PRIOR ART DOCUMENT

### Patent Document

Patent Document 1: Japanese Patent Application Publication No. 2005-082708

### DISCLOSURE OF INVENTION

### Problems to be Solved by the Invention

However, in conventional photocatalyst-containing cleansing agents, when the agents are in environments in which light is applied, the oxidation reaction of the photocatalyst gradually proceeds, and there is a drawback that components themselves of the cleansing agent are decomposed by the photocatalyst. In shops, homes, etc. it is difficult to store the cleansing agent in a light-shielded environment, and as a result, the essential sterilizing, cleansing and deodorant effects of the photocatalyst degrade with the passage of time. Particularly, such a problem tends to occur in liquid cleansing agents.

Therefore, in view of the above-mentioned conventional problem, it is an object of the present invention to provide a cleansing agent for preventing the photocatalyst-containing cleansing agent from deteriorating, and enabling the sterilizing, cleansing and deodorant effects to be maintained.

### Means for Solving the Problem

To solve the above-mentioned problem, the invention provides a cleansing agent characterized by containing a photocatalyst, a fine powder of charcoal that is activated carbon, and cyclodextrin.

Herein, as a principal component, the photocatalyst preferably contains any one or more of titanium oxide, zinc oxide, vanadium oxide, bismuth trioxide, tungstic trioxide, iron oxide, and strontium titanate.

Further, it is suitable that the photocatalyst is an apatite-combined photocatalyst and an apatite complex. The apatite may be fluorapatite or hydroxylapatite.

Furthermore, it is suitable that the cleansing agent further contains an activator. The activator is preferably sodium sesquicarbonate. Alternately, the activator may be selected from the group consisting of any one or mixtures of amino acids, acid salts of amino acids, alkali salts of amino acids, sodium percarbonate, sodium persulfate, aluminium hydroxide, coprecipitates of aluminium hydroxide/magnesium carbonate/calcium carbonate, aluminium magnesium hydroxide, coprecipitates of aluminium hydroxide/magnesium hydroxide, coprecipitates of aluminium hydroxide/sodium bicarbonate, aluminium glycinate, calcium acetate, calcium bicarbonate, calcium borate, calcium carbonate, calcium citrate, calcium gluconate, calcium glycerol phosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dried aluminium hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium aluminometasilicate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, dibasic sodium phosphate, sodium hydroxide, sodium lactate, sodium phthalate, sodium phosphate, sodium polyphosphate, sodium pyrophosphate, sodium succinate, sodium tartrate, sodium tripolyphosphate, synthetic hydrotalcite, tetrapotassium pyrophosphate, tetrasodium pyrophosphate, tripotassium phosphate, trisodium phosphate, trometamol, perborates, carbonates, metasilicates, citric acid, sodium bicarbonate, and calcium silicate.

In addition, the cleansing agent preferably contains an essential oil obtained from one or more natural plants including lavender, rosemary, sweet orange, cedar wood, chamomile, and peppermint. Further, the cleansing agent may contain a moisturizer obtained from one or more natural materials including beeswax, honey, coconut milk powder, seaweed, brown sugar, yoghurt, and egg yolk.

Then, it is suitable that the cleansing agent is one of a shampoo, body shampoo, hand soap, makeup cleansing agent, facial cleanser, and kitchen cleanser.

In addition, it is desirable that a container that stores the cleansing agent is formed of a light-blocking member that prevents light from entering from the outside.

### Advantageous Effect of the Invention

According to the cleansing agent of the invention, by adding a fine powder of charcoal in addition to a photocatalyst, it is possible to inhibit the oxidation reaction of the photocatalyst during storage by the light blocking effect, and to prevent the effects of the cleansing agent from degrading. Further, it is possible to efficiently exert cooperative action of adsorbing excess sebum, pore-clogging dirt, cuticle, waste products, smells, etc. due to the porous structure of charcoal, and further, decomposing the adsorbed materials by the photocatalyst. When the cleansing agent is applied to shampoos, it is also possible to maintain the scalp healthy and also obtain the hair growth effect.

### BRIEF DESCRIPTION OF DRAWINGS

FIG. 1 is a diagram to explain the functions of a photocatalyst and apatite complex contained in a cleansing agent of the invention.

### BEST MODE FOR CARRYING OUT THE INVENTION

An Embodiment of the invention will specifically be described below.

A cleansing agent according to the invention is characterized in that a photocatalyst, fine powder of charcoal that is activated carbon and cyclodextrin are added thereto. As the charcoal, charcoal or activated carbon is particularly suitable.

Herein, the charcoal is charcoal obtained by heating vegetable tissues such as wood in a semi-airtight state to carbonize. Further, the activated carbon is charcoal with high absorptivity obtained by applying chemical or physical treatment (activation).

Charcoal and activated carbon has the property of being porous. In other words, charcoal and activated carbon is comprised of structure provided with an infinite number of pores, and is capable of capturing and adsorbing fine substances in the pores. For example, black charcoal (charcoal obtained by carbonizing wood using an earthen furnace) has a wide surface area ranging from 200 m² to 300 m² per gram. As the surface area is wider, the adsorbability is higher. In activated carbon, the surface area ranges from 900 m2 to 1300 m2 per gram. Therefore, activated carbon is particularly excellent in adsorbability.

When such charcoal and activated carbon is added to a photocatalyst-containing cleansing agent, it is possible to adsorb excess sebum, pore-clogging dirt, cuticle, waste products, smells, etc. in the pores. Then, it is further possible to efficiently exert cooperative action that the photocatalyst decomposes the adsorbed materials.

Meanwhile, a fine powder of charcoal is black (charcoal black). Therefore, by adding a fine powder of charcoal, the liquid color of the cleansing agent is made black, and by its light blocking effect, the oxidation reaction of the photocatalyst during storage is inhibited, while it is possible to prevent the action of the cleansing agent from degrading.

Thus, in the cleansing agent of the invention, by adding a fine powder of charcoal in addition to a photocatalyst, as well as exerting the great sterilizing, cleansing and deodorant effects in cooperation with the photocatalyst, it is possible to prevent the cleansing agent from deteriorating and to maintain the aforementioned effects.

In addition, it is suitable that as a principal component the photocatalyst for use in the invention contains any one or more of titanium oxide, zinc oxide, vanadium oxide, bismuth trioxide, tungstic trioxide, iron oxide, and strontium titanate.

It is desirable that the photocatalyst for use in the cleansing agent of the invention is a complex with apatite. Then, the photocatalyst and apatite complex will be described below.

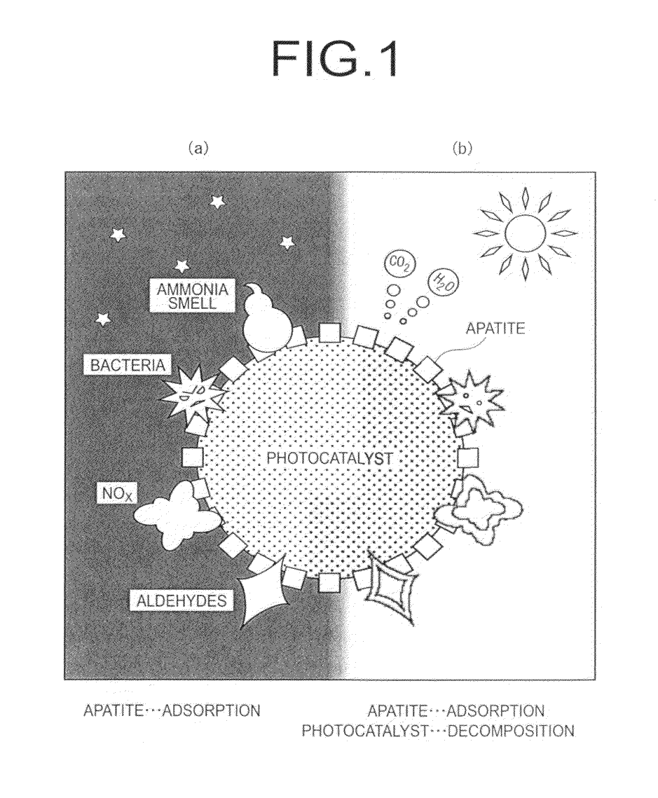

FIG. 1 is a diagram to explain the functions of the photocatalyst and apatite complex contained in the cleansing agent of the invention.

The apatite refers to a group of minerals having the composition of M₁₀(ZO₄)₆X₂ where M, Z and X are arbitrary elements. The principal components of aforementioned M and Z are respectively calcium and phosphorus, and it is possible to replace M, Z and X with various elements relatively with ease. For example, when M contains calcium, Z contains phosphorus and X contains fluorine as principal components, fluorapatite is obtained. Meanwhile, when M contains calcium, Z contains phosphorus and X contains hydroxyl groups, hydroxylapatite is obtained. As the function of apatite, the function of adsorbing and holding hazardous chemical substances, etc. is generally known well. For example, it is possible to adsorb nitrogen oxides, lipid peroxides, ammonia, aldehydes, bacteria such as Escherichia coli, and viruses.

The photocatalyst and apatite complex is generated by combining a photocatalyst and apatite by a method of coating (coating the photocatalyst with the apatite), substitution (substituting the photocatalyst into the crystal structure of the apatite), or the like. Then, by coordinating the action of both the photocatalyst and the apatite to work together, it is possible to efficiently absorb and decompose hazardous organic substances, bacteria, bad-smell substances and the like. For example, when light is applied, the photocatalyst causes extremely strong oxidizability, decomposes a hazardous chemical substance, bacteria and the like brought into contact with photocatalyst, is capable of decomposing into carbon dioxide and the like, but does not have action of attracting the substances, bacteria and the like, or of adsorbing large amounts thereof, and is capable of decomposing only substances brought into contact with the surface of the photocatalyst. Further, the photocatalyst is not able to function unless light is applied. The apatite is excellent in the ability of adsorbing substances, and is capable of adsorbing large amounts, but does not have the ability of decomposing the substances. Therefore, when the adsorption amount exceeds a certain adsorption amount, the apatite is saturated and is not able to adsorb any more.

However, when the photocatalyst and apatite are combined to be a complex, as shown in FIG. 1(a), the complex is capable of adsorbing substances within a period during which light is not applied. Further, as shown in FIG. 1(b), when light is applied, since the photocatalyst decomposes the substances adsorbed by the apatite, the adsorption function is reproduced every time. Furthermore, the photocatalyst needs a certain time period to decompose organic substances, and since the apatite does not allow the adsorbed substances to escape, is capable of reliably decomposing the substances.

In the cleansing agent according to the invention, by including the photocatalyst and apatite complex in charcoal having the above-mentioned action and effect, the cleansing agent is capable of having the excellent sterilizing, cleansing and deodorant effects by cooperative action that the apatite adsorbs hazardous organic substances, bad-smell substances, bacteria and the like and that the photocatalyst decomposes and removes the substances. When the cleansing agent is applied to shampoos, it is possible to maintain the scalp healthy and also obtain the hair growth effect.

In addition, it is suitable that the apatite for use in the invention is fluorapatite or hydroxylapatite.

Silicon dioxide may be further added to the cleansing agent of the invention to reinforce and enhance the above-mentioned action and effect of the photocatalyst and apatite.

In the invention, further, cyclodextrin is added which enhances the above-mentioned action of the photocatalyst and has a distinctive effect. The cyclodextrin is cyclic oligosaccharides that glucoses are bound in cyclic structure, and inside the cyclic structure, has vacancies of the size of the order allowing inclusion of another molecule. The cyclodextrin has the peculiar structure that the exterior of the vacancy is hydrophilic and that the interior thereof is hydrophobic (lipophilic), captures various molecules inside the vacancies, and is capable of forming inclusion complexes. Therefore, conventionally, by using the inclusion function, the cyclodextrin has been used in increasing stability of guest molecules (enclosed molecules), removing bad smells, and different tastes of foods, etc. It was confirmed by experiments that such action of the cyclodextrin drastically enhances the above-mentioned action and effect of the photocatalyst and apatite together with the photocatalyst and apatite. In other words, when the amounts of the photocatalyst and apatite are fixed amounts, with respect to the decomposition and removal rate of hazardous chemical substances, bad-smell substances, bacteria, etc. by the photocatalyst and apatite complex, the decomposition and removal rate in the case where cyclodextrin is added is three to six times that in the case where cyclodextrin is not added.

Among cyclodextrins, the six glucose ring molecule is referred to as α-cyclodextrin, the seven glucose ring molecule is referred to as β-cyclodextrin, and the eight glucose ring molecule is referred to as γ-cyclodextrin. As well as α-cyclodextrin, β-cyclodextrin, and γ-cyclodextrin, the cyclodextrins referred to in the invention include derivatives and analogous compounds of cyclodextrins.

Herein, the effects obtained by adding the cyclodextrin as described above to the cleansing agent of the invention are as described below. First, the cyclodextrin is able to enclose bad-smell substances, hazardous chemical substances and the like as the inclusion. The inclusion is performed quickly, and therefore, the deodorant and cleansing effects appear promptly. Then, the photocatalyst decomposes the substances. Thus, sebum, dirt, smell, bacteria and the like are removed.

In addition, cyclodextrins are also organic substances, and unless the photocatalyst active substance is irradiated with strong light of a certain level or more, do not decompose by photodecomposition action of the photocatalyst active substance. Therefore, even in a state in which the cyclodextrin and photocatalyst coexist, the cyclodextrin does not decompose in a liquid containing a fine powder of charcoal.

Thus, by further adding cyclodextrins, it is possible to enhance action of the photocatalyst.

Further, an activator for activating the photocatalyst effect may be added to the cleansing agent of the invention. For example, it is suitable adding sodium sesquicarbonate, or an activator selected from the group consisting of any one or mixtures of amino acids, acid salts of amino acids, alkali salts of amino acids, sodium percarbonate, sodium persulfate, aluminium hydroxide, coprecipitates of aluminium hydroxide/magnesium carbonate/calcium carbonate, aluminium magnesium hydroxide, coprecipitates of aluminium hydroxide/magnesium hydroxide, coprecipitates of aluminium hydroxide/sodium bicarbonate, aluminium glycinate, calcium acetate, calcium bicarbonate, calcium borate, calcium carbonate, calcium citrate, calcium gluconate, calcium glycerol phosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dried aluminium hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium aluminometasilicate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, dibasic sodium phosphate, sodium hydroxide, sodium lactate, sodium phthalate, sodium phosphate, sodium polyphosphate, sodium pyrophosphate, sodium succinate, sodium tartrate, sodium tripolyphosphate, synthetic hydrotalcite, tetrapotassium pyrophosphate, tetrasodium pyrophosphate, tripotassium phosphate, trisodium phosphate, trometamol, perborates, carbonates, metasilicates, citric acid, sodium bicarbonate, and calcium silicate.

In addition, an essential oil obtained from one or more natural plants including lavender, rosemary, sweet orange, cedar wood, chamomile, and peppermint may be added to the cleansing agent of the invention so as to obtain an aroma and peculiar effect. When the essential oil is beforehand enclosed in the cyclodextrin, it is possible to obtain the aroma in use. Further, as a moisturizer, beeswax, honey, coconut milk powder, seaweed, brown sugar, yoghurt, egg yolk or the like may be added.

Herein, the cleansing agent referred to in the invention indicates cleansing agents used for the human body, and particularly, it is suitable that the agent is a shampoo. Alternately, the agent is anyone of a body shampoo, hand soap, makeup cleansing agent, facial cleanser, and kitchen cleanser.

Herein, it is desirable that a container that stores the cleansing agent is formed of a light-blocking member that prevents light from entering from the outside.

As described above, according to the cleansing agent of the invention, by adding a fine powder of charcoal in addition to a photocatalyst, it is possible to inhibit the oxidation reaction of the photocatalyst during storage by the light blocking effect, and to prevent the effects of the cleansing agent from degrading. Further, it is possible to efficiently exert cooperative action of adsorbing excess sebum, pore-clogging dirt, cuticle, waste products, smells, etc. due to the porous structure of charcoal, and further, decomposing the adsorbed materials by the photocatalyst.

In the above-mentioned description, the Embodiment of the invention is described, but the invention is not limited to the above-mentioned Embodiment, and is capable of being carried into practice with various modifications thereof based on the subject matter of the invention, and the modifications are not excluded from the scope of the invention.

### INDUSTRIAL APPLICABILITY

The present invention relates to a cleansing agent for preventing the photocatalyst-containing cleansing agent from deteriorating, and enabling the sterilizing, cleansing and deodorant effects to be maintained, and has industrial applicability.

1. A cleansing agent containing a photocatalyst, a fine powder of charcoal that is activated carbon, and cyclodextrin.

2. The cleansing agent according to claim 1, wherein the agent further contains silicon dioxide.

3. The cleansing agent according to claim 1, wherein as a principal component, the photocatalyst contains any one or more of titanium oxide, zinc oxide, vanadium oxide, bismuth trioxide, tungstic trioxide, iron oxide, and strontium titanate.

4. The cleansing agent according to claim 3, wherein the photocatalyst is an apatite-combined photocatalyst and an apatite complex.

5. The cleansing agent according to claim 4, wherein the apatite is fluorapatite or hydroxylapatite.

6. The cleansing agent according to claim 5, wherein an activator is selected from the group consisting of any one or mixtures of sodium sesquicarbonate, amino acids, acid salts of amino acids, alkali salts of amino acids, sodium percarbonate, sodium persulfate, aluminium hydroxide, coprecipitates of aluminium hydroxide/magnesium carbonate/calcium carbonate, aluminium magnesium hydroxide, coprecipitates of aluminium hydroxide/magnesium hydroxide, coprecipitates of aluminium hydroxide/sodium bicarbonate, aluminium glycinate, calcium acetate, calcium bicarbonate, calcium borate, calcium carbonate, calcium citrate, calcium gluconate, calcium glycerol phosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dried aluminium hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium aluminometasilicate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, dibasic sodium phosphate, sodium hydroxide, sodium lactate, sodium phthalate, sodium phosphate, sodium polyphosphate, sodium pyrophosphate, sodium succinate, sodium tartrate, sodium tripolyphosphate, synthetic hydrotalcite, tetrapotassium pyrophosphate, tetrasodium pyrophosphate, tripotassium phosphate, trisodium phosphate, trometamol, perborates, carbonates, metasilicates, citric acid, sodium bicarbonate, and calcium silicate.

7. The cleansing agent according to claim 1, wherein the cleansing agent contains an essential oil obtained from one or more natural plants including lavender, rosemary, sweet orange, cedar wood, chamomile, and peppermint.

8. The cleansing agent according to claim 1, wherein the cleansing agent contains a moisturizer obtained from one or more natural materials including beeswax, honey, coconut milk powder, seaweed, brown sugar, yoghurt, and egg yolk.

9. The cleansing agent according to claim 1, wherein the cleansing agent is one of a shampoo, body shampoo, hand soap, makeup cleansing agent, facial cleanser, and kitchen cleanser.

10. The cleansing agent according to claim 9, wherein a container that stores the cleansing agent is formed of a light-blocking member that prevents light from entering from the outside.
